(19) United States
(12) Patent Application Publication
(10) Pub. No.: US 2003/0187067 A1
(43) Pub. Date: Oct. 2, 2003

(54) Use of anethole-dithiolethione for preventing and treating drug-induced tendon toxicity

(76) Inventors: Marie-Odile Christen, Paris (FR); Jean-Michel Warnet, Paris (FR); Patrice Rat, Paris (FR)

Correspondence Address: YOUNG & THOMPSON, 745 SOUTH 23RD STREET 2ND FLOOR, ARLINGTON, VA, 22202

(21) Appl. No.: 10/258,520

(22) PCT Filed: Apr. 25, 2001
(86) PCT No.: PCT/FR01/01268

(30) Foreign Application Priority Data
Apr. 25, 2000 (FR) 00/05247

(51) Int. Cl. A61K 31/21

(52) U.S. Cl. 514/513

(57) ABSTRACT

The invention concerns the use of anethole-dithiolethione for producing a medicine for preventing drug-induced tendon toxicity.

The present invention relates to a new use of anethole-dithiolethione (or anethole trithione).

Quinolones and, in particular, second-generation quinolones, fluoroquinolones, are synthesis antibiotics which, because of their very high level of activity, are very widely used.

These agents are relatively well tolerated; nevertheless, a secondary effect has been recorded since the 1990s. This effect relates to tendinopathies, including cases of tendinitis and tendon ruptures which occur in the majority of cases in the region of the Achilles tendon, but which can also affect the quadriceps tendon or, in the region of the shoulder, the supraspinal tendon. The duration of disablement is, on average, 62 days for simple cases of tendinitis and 98 days for ruptures.

Other molecules seem to have similar effects, such as statins. Some cases of tendinopathies and tendon ruptures have been found with simvastatin (Zocor® or Lodales®), but also with the new recently marketed generations of statins.

This tendon toxicity phenomenon was not well known until now and there were no remedial means or agents.

The constituent cell of the tendon is the tenocyte; in the tendon, these cells are organised in columns between the collagen fibres.

The authors of the present invention have studied, in a cellular tenocyte model, the cytotoxic effect of fluoroquinolones. They then determined, in the model, that anethole-dithiolethione (Sulfarlem®), which was tested during preliminary treatment before being placed in contact with those xenobiotics, protects against this tendon toxicity in a surprising and unique manner, demonstrating the advantages of this molecule in the prevention and treatment of tendon-toxicity induced by drugs such as quinolones or statins.

Therefore, anethole-dithiolethione can be used as a drug administered alone or together with antibiotics or statins to prevent the tendon toxicity induced by xenobiotics such as quinolones or statins.

Therefore, the present invention relates to the use of anethole-dithiolethione in the preparation of a drug intended to prevent and treat tendon toxicity induced by drugs such as quinolones or statins, containing anethole-dithiolethione alone or being in admixture or association with an inductor drug.

Anethole-dithiolethione can be used in a form containing from 10 to 200 mg and can be administered in doses of from 10 to 200 mg/day.

The invention also comprises a method for treating or preventing tendon toxicity induced by drugs such as quinolones or statins, consisting in administering to a patient a drug which contains anethole-dithiolethione which may or may not be associated with an inductor drug.

Results of tests demonstrating the protective effect of anethole-dithiolethione are given below.

The test carried out is a type II MIFALC (Microtitration Fluorimetric Assay on Living Cells) test, that is to say that all of the steps, including detection/disclosure, are carried out on living cells, the test using fluorimetric detection under cold light, in order to achieve the maximum level of detection sensitivity and specificity necessary for the intracellular studies. The tests are carried out on a line of immortalised tenocytes, known as TENO (Teno Cell Line).

Preparation of Cultures

The cultures are produced in 75 cm³ flasks for cell multiplication and in plates having 96 shallow wells for culture.

The tenocytes are placed in suspension with a small amount of trypsin. When the cells are released, the action of the trypsin is blocked by the addition of culture medium containing fetal calf serum. After centrifuging, the residue is taken up by complete medium HAM F-12, then the cells are counted using a Malassez or Thoma hemocytometer. Next, a solution of 27,500 cells/ml is prepared in the complete culture medium and 200 μl are placed in each of the central 60 wells of the microplates having 96 wells. Therefore, there are 5,500 cells per well. The plates are placed in incubation at 37° C. in a humid atmosphere of air/CO₂ (95%/5%). The test is carried out after 48 hours when the cells adhere to the support.

Test

The different fluoroquinolones (or statins) are used in solution ranging from 0.1 μM to 1 mM in an adapted solvent whose harmlessness has been verified beforehand. The tendon toxicity of those molecules was studied according to different protocols: without pretreatment or with pretreatment by anethole-dithiolethione (in a dose of 20 μM), 72 hours before being placed in contact with the xenobiotics; the cells are then placed in culture in microplates directly in suspension in solutions of anethole-dithiolethione.

On the day of the test, after observing the confluence and the microscopic state of the cells, the culture medium is eliminated by turning over the plates and 200 μl of a 20 μMol solution of dihydro-dichlorofluorescein diacetate (H₂DCF-DA), which is a fluorogen probe which penetrates into the cells and remains localised in their cytosol, are distributed in each well; the agent is routinely used to evaluate free radicals produced by the cell.

The plates are then placed in an oven at 37° for 20 minutes.

After eliminating the probe by turning over the plates, the wells are treated with the different solutions of xenobiotics and placed in an oven. The plates are then placed in the fluorometer which is provided with a 535 nm emission filter and a 485 nm excitation filter.

The fluorescence units are read after 40 minutes.

Results

The results are expressed as fluorescence percentages relative to the specimen.

[image]

[image]

1. Use of anethole-dithiolethione in the manufacture of a drug for preventing or treating tendon toxicity induced by an inductive drug.

2. Use according to claim 1 in the manufacture of a drug containing from 10 to 200 mg of anethole-dithiolethione.
